(19) United States
(12) Patent Application Publication
(10) Pub. No.: US 2007/0082328 A1
(43) Pub. Date: Apr. 12, 2007

(54) ROTATABLE TIME VARYING ELECTROMAGNETIC FORCE BIOREACTOR AND METHOD OF USING THE SAME

(76) Inventor: DONNIE RUDD, SUGAR LAND, TX (US)

Correspondence Address: LADAS & PARRY LLP, 224 SOUTH MICHIGAN AVENUE, SUITE 1600, CHICAGO, IL, 60604, US

(21) Appl. No.: 11/535,148

(22) Filed: Sep. 26, 2006

Related U.S. Application Data
(60) Provisional application No. 60/720,803, filed on Sep. 27, 2005.

(51) Int. Cl. C12Q 1/00; C12N 5/06

(52) U.S. Cl. 435/4; 435/325; 435/289.1

(57) ABSTRACT

A rotatable time varying electromagnetic force bioreactor comprising a culture chamber having an interior portion that removably receives a cell mixture and an exterior portion with an electrically conductive coil wrapped around the exterior portion, a motor for rotating the culture chamber, and a time varying electromagnetic force source operatively connected to the electrically conductive coil. In use, the time varying electromagnetic force source delivers a time varying electromagnetic force to the interior portion of the culture chamber exposing the mammalian cells therein to a time varying electromagnetic force thereby expanding the mammalian cells.

### FIELD OF THE INVENTION

The present invention generally relates to a method for expanding mammalian cells. More particularly, the present invention relates to a rotatable time varying electromagnetic force bioreactor, a method for expanding mammalian cells therein, and a composition related thereto.

### BACKGROUND OF THE INVENTION

Cell culture processes have been developed for the growth of single cell bacteria, yeast and molds which are resistant to environmental stresses or are encased with a tough cell wall. Mammalian cell culture, however, is much more complex because mammalian cells are more delicate and have more complex nutrient and other environmental requirements in order to maintain viability and cell growth. Large-scale cultures of bacterial type cells are highly developed and such culture processes are less demanding and are not as difficult to cultivate as mammalian cells. Bacterial cells can be grown in large volumes of liquid medium and can be vigorously agitated without any significant damage. Mammalian cells, on the other hand, cannot withstand excessive turbulent action without damage to the cells and are typically provided with a complex nutrient medium to support growth.

In addition, mammalian cells have other special requirements; in particular most animal cells typically prefer to attach themselves to some substrate surface to remain viable and to duplicate. On a small scale, mammalian cells have been grown in chambers with small microwells to provide surface anchors for the cells. However, cell culture processes for mammalian cells in such microwell chambers generally do not provide sufficient surface area to grow mammalian cells on a sufficiently large scale basis for many commercial or research applications. To provide greater surface areas, microcarrier beads have been developed for providing increased surface areas for the cultured cells to attach. Microcarrier beads with attached cultured cells require agitation in a conventional bioreactor chamber to provide suspension of the cells, distribution of fresh nutrients, and removal of metabolic waste products. To obtain agitation, such bioreactor chambers have used internal propellers or movable mechanical agitation devices which are motor driven so that the moving parts within a chamber cause agitation in the medium for the suspension of the microcarrier beads and attached cells. Agitating the medium may also agitate mammalian cells therein by subjecting them to high degrees of fluid shear stress that can damage the cells and limit ordered assembly of these cells according to cell derived energy. These fluid shear stresses arise, for instance, when the medium has significant relative motion with respect to chamber walls, internal propellers or movable mechanical agitation devices, or other chamber components. Cells may also be damaged in culture chambers with internal moving parts if the cells or beads with cells attached collide with one another or with chamber components.

In addition to the drawbacks of cell damage, bioreactors and other methods of culturing mammalian cells are also very limited in their ability to provide conditions that allow cells to assemble into tissues that simulate the spatial three-dimensional form of actual tissues in an intact organism and at the same time allow cells to multiply at a rate of at least seven times within seven days. Conventional tissue culture processes limit, for similar reasons, the capacity for cultured tissues to, for instance, develop a highly functionally specialized or differentiated state considered crucial for mammalian cell differentiation and secretion of specialized biologically active molecules of research and pharmaceutical interest. Unlike microorganisms, the cells of higher organisms such as mammals form themselves into high order multicellular tissues. Although the exact mechanisms of this self-assembly are not known, in the cases that have been studied thus far, development of cells into tissues has been found to be dependent on orientation of the cells with respect to each other (the same or different type of cell) or other anchorage substrate and/or the presence or absence of certain substances (factors) such as hormones. In summary, no conventional culture process is capable of simultaneously achieving sufficiently low fluid shear stress, sufficient three-dimensional spatial freedom, and for sufficiently long periods for critical cell interactions (with each other or substrates) to allow excellent modeling of in vivo cell and tissue structure, and at the same time, provide accelerated expansion (at least seven times the number of cells per volume that the culture chamber was originally inoculated with), growth in the size of tissue and/or tissue constructs and/or growth in the number of cells, while maintaining the cell three dimensional geometry, and cell-to-cell geometry and support.

For example, U.S. Pat. No. 5,155,035, Wolf et al., provides a method for culturing tissues, tissue constructs, and cells utilizing a perfused bioreactor that overcomes prior problems without subjecting the tissue and/or cells to destructive amounts of shear. The Wolf et al. disclosure, however, provides for a very low rate of production. In fact, the Wolf et al. device, and method of using the same, provides an insufficiently low production rate such that it is not of a substantial commercial value. Other methods provide for mammalian cell cultures in two-dimensions, but without supporting and maintaining the three-dimensional geometry and cell to cell support and geometry that cells exhibit in the in vivo tissue where they naturally reside over a high rate of expansion.

Schwartz et al., U.S. Pat. No. 4,988,623, and Schwartz et al., U.S. Pat. No. 5,026,650, disclose the growth of a variety of both normal and neoplastic mammalian tissues in both mono-culture and co-culture in both batch-fed and perfused rotating wall chambers. The formation of tissues has been supported and maintained by the use of solid matrix in the form of biocompatible polymers and microcarrier beads. The formation of spheroids has been achieved without the support of a matrix. Goodwin et al., In Vitro Cell Dev. Biol. Admin., 33: 366-74 (1997).

The ex vivo growth of human tissue has been largely refractory including the controlled growth induction and three-dimensional spatial organization of the same. Fukucda et al., U.S. Pat. No. 5,328,843, disclosed the use of zones formed between stainless steel having blades to orient neuronal cells or axons, and an electrical potential was employed to enhance axon response. Aebischer, U.S. Pat. No. 5,030,225, disclosed an electrically charged, implantable tubular membrane for generating severed nerves within the human body. Wolf et al., U.S. Pat. No. 6,485,963, utilized electromagnetic force to increase cell growth, but in many cases the cell growth did not occur rapidly enough for needed testing or treatment of a patient.

It is highly desirable, therefore, to have a rotatable time varying electromagnetic force (“TVEMF”) bioreactor that, when in use, not only achieves sufficiently low fluid shear stress, sufficient three-dimensional spatial freedom, and for sufficiently long periods for critical cell interactions (with each other or substrates), but at the same time, provides accelerated expansion while supporting and maintaining essentially the same three dimensional geometry, and cell-to-cell geometry and support as that of the cells natural in vivo environment. It is also highly desirable to have a method for expanding mammalian cells at least seven times the number per volume as were placed in the rotatable TVEMF bioreactor in less than seven days while at the same time supporting and maintaining the cells three dimensional geometry, and cell-to-cell geometry and support.

### SUMMARY OF THE INVENTION

The present invention relates to a rotatable TVEMF bioreactor comprising a substantially cylindrical culture chamber having at least one aperture, an interior portion, and an exterior portion wherein the interior portion defines a space that removably receives a cell mixture; an electrically conductive coil wrapped around the exterior portion of the culture chamber; a motor connected to the culture chamber to rotate the culture chamber about a substantially horizontal axis; and a time varying electromagnetic force source operatively connected to the electrically conductive coil.

The present invention also relates to a method for expanding mammalian cells comprising the steps of providing a culture chamber of a rotatable TVEMF bioreactor, filling the culture chamber with a culture medium, placing a cell mixture containing mammalian cells into the culture chamber and initiating a three-dimensional culture wherein the mammalian cells have a three-dimensional geometry and cell-to-cell support and geometry essentially the same as the cells in vivo, rotating the culture chamber about a substantially horizontal axis at a rotation speed while at the same time exposing the three-dimensional culture to a time varying electromagnetic force, controlling the rotation of the culture chamber to maintain the three-dimensional culture, and continuing rotating the culture chamber until the number of mammalian cell per volume is at least seven times greater than the number of mammalian cells in the cell mixture placed in the culture chamber. Preferably, the method of the present invention has the properties of collocation of the culture medium and the cells, essentially no relative m-notion of the culture medium with respect to the rotatable TVEMF bioreactor, and freedom for three-dimensional spatial orientation of the cells.

Other aspects, features, and advantages of the present invention will be apparent from the following description of the preferred embodiments of the invention given for the purpose of disclosure. This invention may be more fully described by the preferred embodiment(s) as hereinafter described, but is not intended to be limited thereto.

### BRIEF DESCRIPTION OF THE DRAWINGS

In the drawings,

FIG. 1 is a cross-sectional elevated side view of a preferred embodiment of a rotatable TVEMF-bioreactor;

FIG. 2 is a side perspective of a preferred embodiment of a rotatable TVEMF-bioreactor;

FIG. 3 schematically illustrates a preferred embodiment of a culture medium flow loop;

FIG. 4 is the orbital path of a typical cell in a non-rotating reference frame;

FIG. 5 is a graph of the magnitude of deviation of a cell per revolution; and

FIG. 6 is a representative cell path as observed in a rotating reference frame of the culture medium.

### DETAILED DESCRIPTION OF THE DRAWINGS

In the simplest terms, a rotatable TVEMF bioreactor comprises a chamber that, in operation, can be controllably rotated about a substantially horizontal axis, and has an interior portion and an exterior portion. The interior portion of the culture chamber defines a space that may removably receive a cell mixture. An electrically conductive coil is wrapped around the exterior portion of the culture chamber. A TVEMF source is operatively connected to the electrically conductive coil so that, in use, the TVEMF source delivers a TVEMF to the interior portion of the culture chamber and to the three-dimensional culture to expand the cells therein. The culture chamber has at least one aperture so that, when in use, the cell mixture may be placed into the interior portion of the chamber. The aperture may also preferably be used for the exchange of culture medium and the removal of cell samples, and preferably the aperture is fitted for use with a syringe.

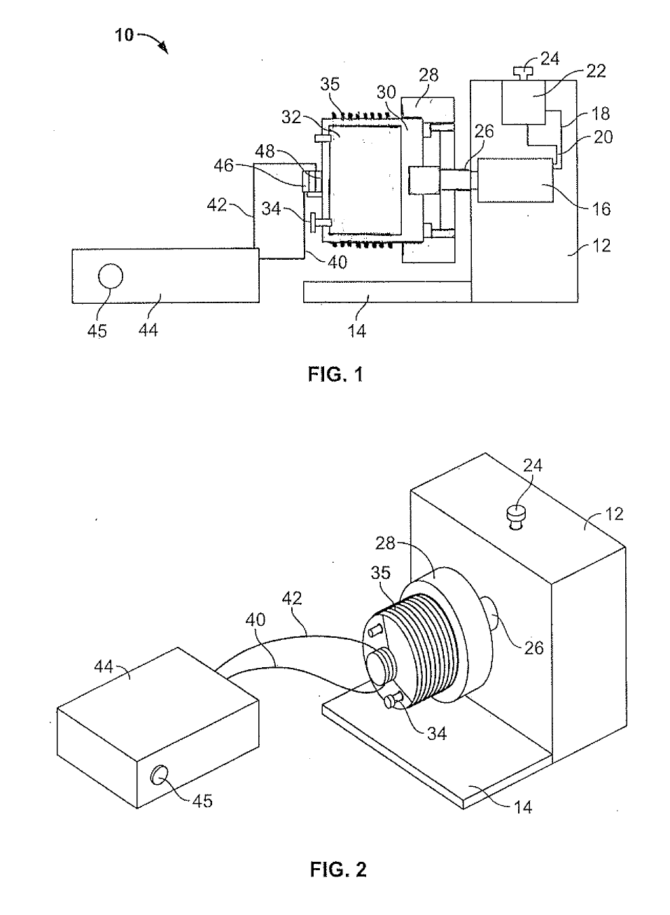

In the drawings, referring now to FIG. 1, FIG. 1 is a cross sectional elevated side view of a preferred embodiment of a rotatable TVEMF bioreactor 10. In this preferred embodiment a motor housing 12 is supported by a base 14. A motor 16 is affixed inside the motor housing 12 and connected by a first wire 18 and a second wire 20 to a control box 22 that houses a control device therein whereby the speed of the motor 16 can be incrementally controlled by turning the control knob 24. Extending from the motor housing 12 is a motor shaft 26. A rotatable mounting 28 removably receives a rotatable TVEMF bioreactor holder 30 that removably receives a culture chamber 32 preferably disposable and substantially cylindrical, which is affixed, preferably removably, within the rotatable TVEMF bioreactor holder 30, preferably by a screw 34. The culture chamber 32 is mounted, preferably removably, to the rotatable mounting 28. The rotatable mounting 28 is received by the motor shaft 26. When the control knob 24 is turned on, the culture chamber 32 is rotated about a substantially horizontal axis at a speed that preferably fosters, supports, and maintains the cells three-dimensional geometry and cell to cell support and geometry while at the same time preventing cell collision with the interior portion of the rotatable TVEMF bioreactor and with other cells.

The culture chamber of the rotatable TVEMF bioreactor of the present invention may preferably be disposable wherein it can be discarded and a new one used in later cell cultures. The rotatable TVEMF bioreactor may also preferably be sterilized, for instance in an autoclave, after each use and re-used for later cell cultures. A disposable culture chamber could be manufactured and packaged in a sterile environment thereby enabling it to be used by the medical or research professional much the same as other disposable medical devices are used.

FIG. 1 also illustrates an electrically conductive coil 35 wrapped around the exterior portion of the culture chamber 32. The electrically conductive coil may preferably be made of any electrically conductive material that conducts electricity including, but not limited to, the following conductive materials; silver, gold, copper, aluminum, iron, lead, titanium, uranium, a ferromagnetic metal, and zinc, or a combination thereof. The electrically conductive coil may also preferably comprise salt water. The electrically conductive coil may also preferably be a solenoid. Furthermore, the electrically conductive coil may preferably be contained in an electric insulator comprising, but not limited to, rubber, plastic, silicones, glass, and ceramic. The electrically conductive coil may be wrapped around the exterior portion of the culture chamber, and therefore, the culture chamber supports a shape of the electrically conductive coil, preferably having a substantially oval cross-section, more preferably a substantially elliptical cross-section, and most preferably a substantially circular cross-section. The electrically conductive coil that is integral with a disposable culture chamber is installed into the TVEMF bioreactor along with the disposable culture chamber and operatively connected to the TVEMF source. When the disposable culture chamber is discarded, the electrically conductive coil is discarded therewith.

At a first end a first conductive wire 40 and a second conductive wire 42, both of which are integral with the electrically conductive coil 35, are operatively connected to a TVEMF source 44 having a source knob 45 which, in use, can be turned on to generate a TVEMF. At a second end the wires 40, 42 are connected to at least one ring to facilitate the rotation of the electrically conductive coil 35, preferably a first ring 46 and a second ring 48 respectively. When the control knob 24 is turned on, the culture chamber 32 and the electrically conductive coil 35 are rotated simultaneously. Furthermore, the electrically conductive coil 35 remains affixed to, and encompassing, the culture chamber 32, while at the same time supplying a TVEMF to the cells in the rotatable TVEMF bioreactor 32.

FIG. 2 illustrates a side perspective view of the rotatable TVEMF bioreactor wound by an electrically conductive coil 35 also depicted in the preferred embodiment of FIG. 1 wherein the electrically conductive coil 35 is wrapped around, and encompassing, a culture chamber 32.

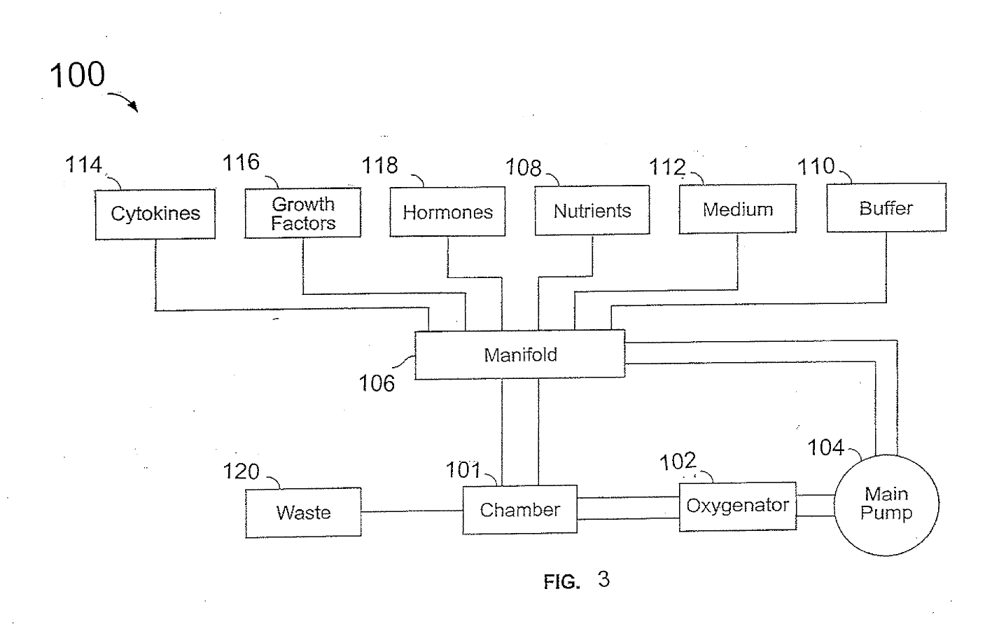

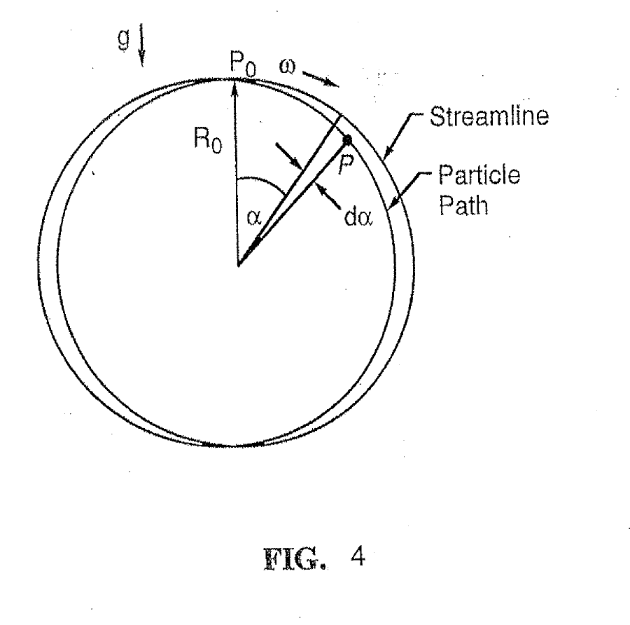

The culture clamber of a rotatable TVEMF bioreactor may preferably be fitted with a culture medium flow loop 100 preferably for the support of respiratory gas exchange in, supply of nutrients in, and removal of metabolic waste products from a three-dimensional TVEMF culture. A preferred embodiment of a culture medium flow loop 100 is illustrated in FIG. 3, having a culture chamber 101, an oxygenator 102, an apparatus for facilitating the directional flow of the culture medium, preferably by the use of a main pump 104, and a supply manifold 106 for the selective input of culture medium requirements such as, but not limited to) nutrients 108, buffers 110, fresh medium 112, cytokines 114) growth factors 116, and hormones 118. In this preferred embodiment, the main pump 104 provides fresh culture medium from the supply manifold 106 to the oxygenator 102 where the culture medium is oxygenated and passed through the culture chamber 101. The waste in the spent culture medium from the culture chamber 101 is removed, preferably by the main pump 104, and delivered to the waste 120 and the remaining volume of culture medium not removed to the waste 120 is returned to the supply manifold 106 where it may preferably receive a fresh charge of culture medium requirements before recycling by the pump 104 through the oxygenator 102 to the culture chamber 101.

In this preferred embodiment of a culture medium flow loop 100, adjustments are made in response to chemical sensors (not shown) that maintain constant conditions within the culture chamber 101. Controlling carbon dioxide pressures and introducing acids or bases corrects pH. Oxygen, nitrogen, and carbon dioxide are dissolved in a gas exchange system (not shown) in order to support cell respiration. The culture medium flow loop 100 adds oxygen and removes carbon dioxide from a circulating gas capacitance. Although FIG. 3 is one preferred embodiment of a culture medium flow loop that nay be used in the present invention, the invention is not intended to be so limited. The input of culture medium requirements such as, but not limited to, oxygen, nutrients, buffers, fresh medium, cytokines, growth factors, and hormones into a rotatable TVEMF bioreactor can also be performed manually, automatically, or by other control means, as can be the control and removal of waste and carbon dioxide.

As various changes could be made in rotatable TVEMF bioreactors such as are contemplated in the present invention, without departing from the scope of the invention, it is intended that all matter contained herein be interpreted as illustrative and not limiting.

### DETAILED DESCRIPTION OF THE INVENTION

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “TVEMF” refers to “time varying electromagnetic force”. As discussed above, the TVEMF of this invention is in a delta wave, more preferably a differential square wave, and most preferably a square wave (following a Fourier curve). The TVEMF is preferably selected from one of the following: (1) a TVEMF with a force amplitude less than 100 gauss and slew rate greater than 1000 gauss per second, (2) a TVEMF with a substantially low force amplitude bipolar square wave at a frequency less than 100 Hz., (3) a TVEMF with a substantially low force amplitude square wave with less than 100% duty cycle, (4) a TVEMF with slew rates greater than 1000 gauss per second for duration pulses less than 1 ms, (5) a TVEMF with slew rate bipolar delta function-like pulses with a duty cycle less than 1%, (6) a TVEMF with a force amplitude less than 100 gauss peak-to-peak and slew rate bipolar delta function-like pulses and where the duty cycle is less than 1%, (7) a TVEMF applied using a solenoid coil to create uniform force strength throughout the three-dimensional culture, (8) and a TVEMF applied utilizing a flux concentrator to provide spatial gradients of magnetic flux and magnetic flux focusing within the three-dimensional culture. The range of frequency in oscillating electromagnetic force strength is a parameter that may be selected for achieving the desired stimulation of the cells in the three-dimensional culture. However, these parameters are not meant to be limiting to the TVEMF of the present invention, as such may vary based on other aspects of this invention. The TVEMF may be measured for instance by standard equipment such as an EN131 Cell Sensor Gauss Meter.

As used throughout this application, the term “electrically conductive coil,” refers to any electrically conductive material that conducts electricity including, but not limited to, the following conductive materials; silver, gold, copper, aluminum, iron, lead, titanium, uranium, a ferromagnetic metal, and zinc, or a combination thereof. The electrically conductive coil may also preferably comprise salt water. The electrically conductive coil may also preferably be a solenoid. Furthermore, the electrically conductive coil may preferably be contained in an electric insulator comprising, but not limited to, rubber, plastic, silicones, glass, and ceramic. The electrically conductive coil may be wrapped around the exterior portion of the culture chamber of the rotatable TVEMF bioreactor, and therefore, preferably the culture chamber supports a shape of the electrically conductive coil, preferably having a substantially oval cross-section, more preferably a substantially elliptical cross-section, and most preferably a substantially circular cross-section. The culture chamber supports a shape of the electrically conductive coil preferably because the shape of the culture chamber and the shape of the electrically conductive coil are substantially similar. By “wrapped around,” it is intended that the electrically conductive coil encompasses the culture chamber so that preferably, in operation, a substantially uniform TVEMF is delivered to the interior portion of the culture chamber and the cells therein. By “encompasses” it is meant that the electrically conductive coil surrounds the culture chamber, and in use, delivers a preferably substantially uniform TVEMF to the interior portion of the culture chamber.

As used throughout this application, the term “cells” refers to a cell in any form, for example, individual cells, tissue, cell aggregates, cells pre-attached to cell attachment substrates for instance microcarrier beads, tissue-like structures, or intact tissue resections. The cells that call be used in this invention are from mammalian tissue, preferably human, more preferably adult stein cells, most preferably peripheral blood adult stem cells, and even more preferably mesenchymal cells. Other mammalian cells that can be used in the method of the present invention preferably include, but are not limited to, heart, liver, hematopoietic, skin, muscle, intestinal, pancreatic, central nervous system, cartilage, connective pulmonary, spleen, bone, and kidney.

As used throughout this application, the term “rotatable TVEMF bioreactor” is meant to comprise a motor connected to a culture chamber with an interior portion and an exterior portion and further having an electrically conductive coil wrapped around the exterior portion of the culture chamber. A TVEMF source is operatively connected to the electrically conductive coil. In use, a rotatable bioreactor may be rotated and preferably the rotation fosters, supports, and maintains a three-dimensional cell culture, as described for instance in the Description of the Invention, above. In addition TVEMF may be generated by the TVEMF source, and at an appropriate gauss level, may preferably be delivered to the interior portion of the culture chamber via the electrically conductive coil. The volume of the rotatable TVEMF bioreactor is preferably of from about 15 ml to about 2 L. See for instance FIGS. 1 and 2 herein for examples (not meant to be limiting) of a rotatable TVEMF bioreactor of the present invention.

The culture chamber of a rotatable TVEMF bioreactor has rotatable chamber walls so that, in operation, the chamber walls are set into motion relative to the culture medium so that there is essentially no fluid stress sheer in the culture medium. The culture chamber also has at least one aperture for the addition and/or removal of culture medium and/or the cell mixture or portions thereof including, but not limited to, the exchange of culture medium (preferably with additives), and the removal of samples of cells and culture medium containing metabolic waste. The term “removably receives” is intended to refer to the exchange of the cell mixture, culture medium or any portions thereof, and any additives from and to the culture chamber. The culture chamber of the rotatable TVEMF bioreactor is substantially horizontally disposed. The culture chamber is also substantially cylindrical with two ends, and is capable of rotation about a substantially horizontal axis. The culture chamber of the rotatable TVEMF bioreactor of the present invention preferably provides for expansion of cells without perfusion of culture medium over the cells. Without being bound by theory, the rotatable TVEMF, bioreactor provides for the expansion of cells for several days or more, while at the same time, fostering, supporting, and maintaining the cells intricate three-dimensional geometry and cell-to-cell support and geometry.

As used throughout this application, the term “cell mixture” and similar terms, refers to a mixture of cells, preferably with another substance including, but not limited to, culture medium (with and without additives), plasma, buffer, and preservatives. The cell mixture may also preferably solely comprise cells.

As used throughout this application, the term “three-dimensional culture,” refers to the cells in the culture chamber during the process of cell expansion in the culture chamber. The cells in the three-dimensional culture have a three-dimensional geometry and cell-to-cell interactions and geometry fostered, supported, and maintained in the culture chamber by the interactions between the cells in the culture medium. The cells in the three-dimensional culture have essentially the same three-dimensional geometry and cell-to-cell support and geometry as the cells in vivo, in the tissue in which they are found in the mammalian body. Three-dimensional tissue and/or tissue-like structures can also develop from the cells and be sustained and further expanded in the three-dimensional culture.

As used throughout this application, the term “operatively connected,” and similar terms, is intended to mean that the TVEMF source can be connected to the culture chamber in a manner such that in operation, the TVEMF source imparts a TVEMF to the interior portion of the culture chamber of a rotatable TVEMF bioreactor and the cells contained therein. The TVEMF source is operatively connected to the electrically conductive coil and the culture chamber therefore if it is integral with the electrically conductive coil of the culture chamber of the rotatable TVEMF bioreactor. The TVEMF source is operatively connected if, in use, it can impart a TVEMF to the interior portion of the culture chamber.

As used throughout this application, the term “exposing,” and similar terms, refers to the process of supplying a TVEMF to cells contained in the interior portion of the culture chamber of a rotatable TVEMF bioreactor. In operation, a TVEMF source is turned on and set at a preferred gauss range and a preferred waveform so that the same is delivered via the TVEMF source to an electrically conductive coil, wrapped around the exterior portion of the culture chamber of the rotatable TVEMF bioreactor. The TVEMF is then delivered to the cells in the three-dimensional culture in the culture chamber thus exposing the cells to the TVEMF, preferably a substantially uniform TVEMF.

As used throughout this application, the term “culture medium” and similar terms, refers to a liquid comprising, but not limited to, growth medium and nutrients, which is meant for the sustenance of cells over time. The culture medium may be enriched with any of the following, but is not limited thereto; growth medium, buffers, growth factors, hormones, and cytokines. The culture medium is supplied to the cells for suspension within the culture chamber of the rotatable TVEMF bioreactor and to support expansion. The culture medium may preferably be mixed with the cells before being added to the culture chamber of the rotatable TVEMF bioreactor thus making a preferred cell mixture, or may preferably be added to the culture chamber before the cells are added thereby mixing the culture medium and the cells in the rotatable TVEMF bioreactor. The culture medium and the cells combination is referred to as a three-dimensional culture when located in the rotatable TVEMF bioreactor and when the expansion process has begun, and/or after TVEMF has been delivered thereto. The culture medium may preferably be enriched and/or refreshed during expansion as needed. Waste contained in the culture medium, as well as culture medium itself may preferably be removed from the three-dimensional TVEMF culture during expansion as needed. Waste contained in the spent culture medium can be, but is not limited to, metabolic waste, dead cells, and other toxic debris. The culture medium can preferably be enriched with oxygen and preferably has oxygen, carbon dioxide, and nitrogen carrying capabilities.

As used throughout this applications, the term, “placing,” and similar terms, refers to the process of suspending cells in culture medium before adding the cell mixture to the rotatable TVEMF bioreactor. The term “placing,” may also refer to adding cells to culture medium that is already present in the rotatable TVEMF bioreactor. Cells can be placed into the rotatable TVEMF bioreactor along with cell attachment substrates such as microcarrier beads.

As used throughout this application, the term “expansion,” and similar terms, refers to the process of increasing the number of cells in a rotatable TVEMF bioreactor by rotating the cell mixture containing culture chamber while at the same t i m e exposing the three-dimensional culture to a TVEMF. Preferably the cells are expanded to more than seven times their original numbered The expansion of cells in a rotatable TVEMF bioreactor according to the present invention provides for cells that maintain, or have the same or essentially the same, three-dimensional geometry and cell-to-cell support and cell-to-cell geometry as the cells prior to expansion, preferably substantially the same geometry and cell to cell interactions as the cells display in the natural setting or tissue where they naturally exist in the mammalian body. Other aspects of expansion may also provide the exceptional characteristics of the cells of the present invention. Not to be bound by theory, expansion not only provides for high concentrations of cells that maintain their three-dimensional geometry and cell-to-cell support, but also supports and maintains the growth of three-dimensional tissues and tissue-like structures.

As used throughout this application, the term “cell-to-cell geometry” refers to the geometry of cells including the spacing, distance between, and physical relationship of the cells relative to one another. For instance, expanded cells, including those of tissues, cell aggregates, and tissue-like structures, of this invention stay in relation to each other as in the natural setting, the tissue in which the cells naturally occur in vivo; the mammalian body. The expanded cells are within the bounds of natural spacing between cells, in contrast to for instance two-dimensional expansion chambers, where such spacing is not preserved over time and expansion.

As used throughout this application, the term cell-to-cell support” refers to the support one cell provides to an adjacent cell. For instance, tissues, cell aggregates, tissue-like structures, and cells maintain interactions such as chemical, hormonal, neural (where applicable/appropriate) with other cells in the body. In the present invention, these interactions are maintained within normal functioning parameters, meaning they do not for instance begin to send toxic or damaging signals to other cells (unless such would be done in the natural cellular and tissue environment).

As used throughout this application, the term “three-dimensional geometry” refers to the geometry of cells in a three-dimensional state (same as or very similar to their natural state), as opposed to two-dimensional geometry for instance as found in cells grown in a Petri dish, where the cells become flattened and/or stretched. Not to be bound by theory, but the three-dimensional geometry of the cells is maintained, supported, and preserved such that the cell can develop into three-dimensional cell aggregates, tissues and/or tissue-like structures in the three-dimensional culture of the rotatable TVEMF bioreactor. Furthermore, tissues can also be expanded in the rotatable TVEMF bioreactor, while at the same time, maintaining the three-dimensional geometry, and cell-to-cell support and geometry.

For each of the above three definitions, relating to maintenance of “cell-to-cell support” and “cell-to-cell geometry” and “three-dimensional geometry” of the cells of the present invention, the term “essentially the same” and “substantially the same,” means that natural geometry and support are provided in expansion, so that the cells are not changed in such a way as to be for instance dysfunctional, toxic or harmful to other cells. Rather, the cells of the present invention, during and after expansion, mimic the in vivo situation.

In operation, cells are placed into the culture chamber of the rotatable TVEMF bioreactor. In one preferred embodiment, the culture chamber is rotated over a period of time, while at the same time a TVEMF is generated in the culture chamber by the TVEMF source. By “while at the same time,” it is intended that the initiation of the delivery of the TVEMF may be before, concurrent with, or after rotation of the culture chamber is intimate. Upon completion of the period of time, the expanded cells are removed from the culture chamber. In a more complex rotatable TVEMF bioreactor, a culture medium enriched with culture medium requirements preferably including, but not limited to, growth medium, buffer, nutrients, hormones, cytokines, and growth factors, which provides sustenance to the cells, can be periodically refreshed and removed.

In use, the present invention provides a stabilized culture environment into which cells may be introduced, suspended, assembled, grown, and maintained with improved retention of delicate three-dimensional structural integrity by simultaneously minimizing the fluid shear stress, providing three-dimensional freedom for cell and substrate spatial orientation, and increasing localization of cells in a particular spatial region for the duration of the expansion. In use, the present invention also provides these three criteria (hereinafter referred to as “the three criteria above”), and at the same time, exposes the cells to a TVEMF. Of particular interest to the present invention is the dimension of the culture chamber, the sedimentation rate of the cells, the rotation rate, the external gravitational field, and the TVEMF.

The stabilized culture environment referred to in the operation of present invention is that condition in the culture medium, particularly the fluid velocity gradients, prior to introduction of cells, which will support a nearly uniform suspension of cells upon their introduction thereby creating a three-dimensional culture upon addition of the cells. In a preferred embodiment, the culture medium is initially stabilized into a near-solid body horizontal rotation about an axis within the confines of a similarly rotating chamber wall of a rotatable TVEMF bioreactor. The chamber walls are set in motion relative to the culture medium so as to initially introduce essentially no fluid stress shear field therein. Cells are introduced to, and move through, the culture medium in the stabilized culture environment thus creating a three-dimensional culture. The cells move under the influence of gravity, centrifugal, and coriolus forces, and the presence of cells within the culture medium of the three-dimensional culture induces secondary effects to the culture medium. The significant motion of the culture medium with respect to the culture chamber, significant fluid shear stress, and other fluid motions, is due to the presence of these cells within the culture medium.

In most cases the cells with which the stabilized culture environment is primed sediment at a slow rate preferably under 0.1 centimeter per second. It is therefore possible, at this early stage of the three-dimensional culture, to select from a broad range of rotational rates (preferably of from about 2 to about 30 RPM) and chamber diameters (preferably of from about 0.5 to about 36 inches). Preferably) the slowest rotational rate is advantageous because it minimizes equipment wear and other logistics associated with handling of the three-dimensional culture.

Not to be bound by theory, rotation about a substantially horizontal axis with respect to the external gravity vector at an angular rate optimizes the orbital path of cells suspended within the three-dimensional culture. In operation, the cells expand to form a mass of cell aggregates, three-dimensional tissues, and/or tissue-like structures, which increase in size as the three-dimensional culture progresses. The progress of the three-dimensional culture is preferably assessed by a visual, manual, or automatic determination of an increase in the diameter of the three-dimensional cell mass in the three-dimensional culture. An increase in the size of the cell aggregate, tissue, or tissue-like structure in the three-dimensional culture may require appropriate adjustment of the rotation speed in order to optimize the particular paths. The rotation of the culture chamber optimally controls collision frequencies, collision intensities, and localization of the cells in relation to other cells and also the limiting boundaries of the culture chamber of the rotatable TVEMF bioreactor. In order to control the rotation, if the cells are observed to excessively distort inwards on the downward side and outwards on the upwards side then the revolutions per minute (“RPM”) may preferably be increased. If the cells are observed to centrifugate excessively to the outer walls then the RPM may preferably be reduced. Not to be bound by theory, as the operating limits are reached, in terms of high cell sedimentation rates or high gravity strengths, the operator may be unable to satisfy both of these conditions and may be forced to accept degradation in performance as measured against the three criteria above.

The cell sedimentation rate and the external gravitations field place a lower limit on the fluid shear stress obtainable, even within the operating range of the present invention, due to gravitationally induced drift of the cells through the culture medium of the three-dimensional culture. Calculations and measurements place this minimum fluid shear stress very nearly to that resulting from the cells' terminal sedimentation velocity (through the culture medium) for the external gravity field strength. Centrifugal and coriolus induced motion [classical angular kinematics provide the following equation relating the Coriolus force to an object's mass (m), its velocity in a rotating frame (vᵣ) and the angular velocity of the rotating frame of reference (□): F_{Coriolis}=−2 m (w x vᵣ)] along with secondary effects due to cell and culture medium interactions, act to further degrade the fluid shear stress level as the cells expand.

Not to be bound by theory, but as the external gravity field is reduced, much denser and larger three-dimensional structures can be obtained. In order to obtain the minimal fluid shear stress level it is preferable that the culture chamber be rotated at substantially the same rate as the culture medium. Not to be bound by theory, but this minimizes the fluid velocity gradient induced upon the three-dimensional culture. It is advantageous to control the rate and size of tissue formation in order to maintain the cell size (and associated sedimentation rate) within a range for which the rate of expansion is able to satisfy the three criteria above. However, preferably, the velocity gradient and resulting fluid shear stress may be intentionally introduced and controlled for specific research purposes such as studying the effects of shear stress on the three-dimensional cell aggregates. In addition, transient disruptions of the expansion process are permitted and tolerated for, among other reasons, logistical purposes during initial system priming, sample acquisition, system maintenance, and culture termination.

Rotating cells about an axis substantially perpendicular to gravity can produce a variety of sedimentation rates, all of which according to the present invention remain spatially localized in distinct regions for extended periods of time ranging from seconds (when sedimentation characteristics are large) to hours (when sedimentation differences are small). Not to be bound by theory, but this allows these cells sufficient time to interact as necessary to form multi-cellular structures and to associate with each other in a three-dimensional culture. Preferably, cells undergo expansion for at least 4 days, more preferably from about 7 days to about 14 days, most preferably from about 7 days to about 10 days, even more preferably about 7 days. Preferably, TVEMF-expansion may continue in a rotatable TVEMF bioreactor to produce a concentration of cells per volume that is at least 7 times the original concentration of cells per volume that were placed in the rotatable TVEMF bioreactor.

Culture chamber dimensions also influence the path of cells in the three-dimensional culture of the present invention. A culture chamber diameter is preferably chosen which has the appropriate volume, preferably of from about 15 ml to about 2 L for the intended three-dimensional culture and which will allow a sufficient seeding density of cells. Not to be bound by theory, but the outward cells drift due to centrifugal force is exaggerated at higher culture chamber radii and for rapidly sedimenting cells. Thus, it is preferable to limit tile maximum radius of the culture chamber as a function of the sedimentation properties of the tissues anticipated in the final three-dimensional culture stages (when the largest cell aggregates with high rates of sedimentation have formed).

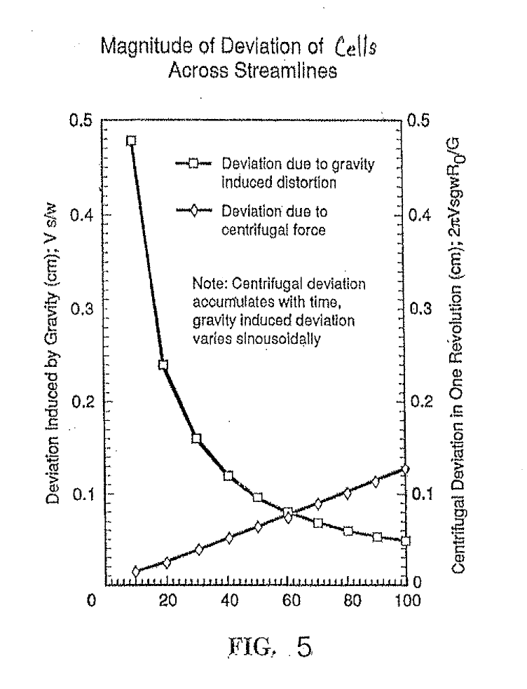

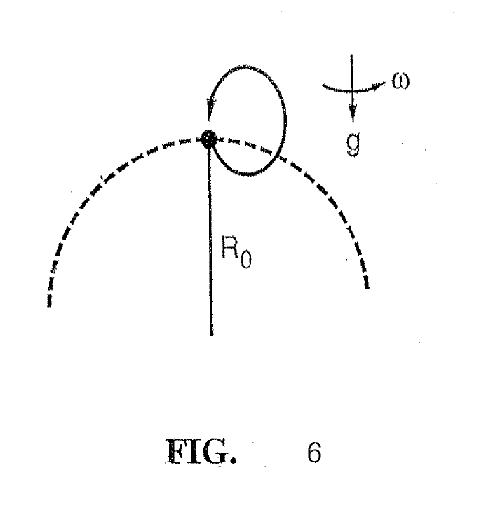

Tile path of the cells in the three-dimensional culture has been analytically calculated incorporating the cell motion resulting from gravity, centrifugation, and coriolus effects. A computer simulation of these governing equations allows the operator to model the process and select parameters acceptable (or optimal) for the particular planned three-dimensional culture. FIG. 4 shows the typical shape of the cell orbit as observed from the external (non-rotating) reference frame. FIG. 5 is a graph of the radial deviation of a cell from the ideal circular streamline plotted as a function of RPM (for a typical cell sedimenting at 0.5 cm per second terminal velocity). This graph (FIG. 5) shows the decreasing amplitude of the sinusoidally varying radial cells deviation as induced by gravitational sedimentation. FIG. 5 also shows increasing radial cells deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the expansion of cells of a given sedimentation rate in a given external gravity field strength. Not to be bound by theory, but as a typical three-dimensional culture is expanded the tissues, cell aggregates, and tissue-like structures increase in size and sedimentation rate, and therefore, the rotation rate may preferably be adjusted to optimize the same.

In the three-dimensional culture, the cell orbit (FIG. 4) from the rotating reference frame of the culture medium is seen to move in a nearly circular path under the influence of the rotating gravity vector (FIG. 6). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the present invention provides cells of differing sedimentation properties with sufficient time to interact mechanically and through soluble chemical signals. In operation, the present invention provides for sedimentation rates of preferably from about 0 cm/second up to 10 cm/second.

Furthermore, in operation the culture chamber of the present invention has at least one aperture preferably for the input of fresh culture medium and a cell mixture and the removal of a volume of spent culture medium containing metabolic waste, but not limited thereto. Preferably, the exchange of culture medium can also be via a culture medium loop wherein fresh or recycled culture medium may be moved within the culture chamber preferably at a rate sufficient to support metabolic gas exchange, nutrient delivery, and metabolic waste product removal. This may slightly degrade the otherwise quiescent three-dimensional culture. It is preferable, therefore, to introduce a mechanism for the support of preferred components including, but not limited to, respiratory gas exchange, nutrient delivery, growth factor delivery to the culture medium of the three-dimensional culture, and also a mechanism for metabolic waste product removal in order to provide a long term three-dimensional culture able to support significant metabolic loads for periods of hours to months.

The present invention exposes the cells to a TVEMF that not only provides for high concentrations of cells that maintain their three-dimensional geometry and cell-to-cell support but in addition, may affect some properties of cells during expansion, for instance up-regulation of genes promoting growth, or down regulation of genes preventing growth. The electromagnetic field is generated by a TVEMF source. In operation, the electrically conductive coil of a rotatable TVEMF bioreactor is rotatable with the culture chambers meaning about the same axis as the culture chamber and in the same direction. Also, the electrically conductive coil is fixed in relation to a culture chamber of a rotatable perfused TVEMF-bioreactor. The electrically conductive coil may be integral with, meaning affixed to and wrapped around the exterior portion of the culture chamber of the electrically conductive coil of the culture chamber of the rotatable TVEMF bioreactor. The TVEMF source is operatively connected to the rotatable TVEMF bioreactor.

In addition to the qualitatively unique cells that are produced by the operation of the present invention, not to be bound by theory, an increased efficiency with respect to utilization of the total culture chamber volume for cell and tissue culture may be obtained due to the substantially uniform homogeneous suspension achieved. Advantageously, therefore, the present invention, in operation, provides an increased number of cells in the same rotatable TVEMF bioreactor with less human resources. Many mammalian cell types may be utilized in this method. Fundamental cell and tissue biology research as well as clinical applications requiring accurate in vitro models of in vivo cell behavior are applications for which the present invention and method of using the same provides an enhancement because, as indicated above and throughout this application, the expanded cells and tissue of the present invention have essentially the same three-dimensional geometry and cell-to-cell support and cell-to-cell geometry as naturally-occurring, non-expanded cells and tissue. The method of the present invention provides these three criteria above in a manner heretofore not obtained and optimizes a three-dimensional culture, and at the same time, facilitates and supports expansion such that a sufficient expansion (increase in number per volume, diameter in reference to tissue, or concentration) is detected in a sufficient amount of time. The present invention is well adapted to carry out the objects and obtain the ends and advantages mentioned herein, as well as those inherent therein. Without departing from the scope of the invention, it is intended that all matter contained herein be interpreted as illustrative and not limiting.

### Operative Method

In operation, a rotatable TVEMF bioreactor preferably having a culture chamber of from 15 ml to 2 L, is completely filled with the appropriate culture medium, preferably supplemented with albumin (5%) and also preferably G-CSF, for the cells to be expanded, with room only for any intended additional volumes of culture medium, cells, and/or other preferred components of the culture medium of the intended three-dimensional culture. Preferably a controlled environment incubator completely surrounds the rotatable TVEMF bioreactor and is preferably set for about 5% CO₂ and about 21% oxygen, and the temperature is preferably of from about 26° C. to about 41° C., and more preferably about 37° C.±2° C.

Initially, a stabilized culture environment is created in the culture medium. The rotation may preferably begin at about 10 RPM, the slowest rate that produces a microcarrier bead orbital trajectory in which the heads do not accumulate appreciably at the chamber walls either by gravitational induced settling or by rotationally induced centrifugation. In this way, the rotatable TVEMF bioreactor produces the minimal fluid velocity gradients and fluid shear stresses in the three-dimensional culture.

Cell attachment substrates are preferably introduced either simultaneously or sequentially with cells into the culture chamber to give an appropriate density, preferably 5 mg of cell attachment substrate per ml of culture medium, and preferably the cell attachment substrate for the anchorage dependent cells are microcarrier heads. The cell mixture is preferably injected into the stabilized culture environment through an aperture in the culture chamber, preferably over a short period of time, preferably 2 minutes, so as to minimize cell damage while passing through the delivery system. Preferably, the cell mixture and/or the cell attachment substrate, if used, is delivered via a syringe. The culture medium is then rotated about a substantially horizontal axis.

After injection of the cells is complete, the culture chamber is quickly returned to initial rotation, preferably in less than one (1) minute, preferably 10 RPM, thereby returning the fluid shear stress to the minimal level obtainable for the cells. During the initial loading and attachment phase, the cells are allowed to equilibrate for a short period of time, preferably of from 2 hours to 4 hours, more preferably for a time sufficient for transient flows to dampen out.

As the expansion of the three-dimensional culture progresses the size and sedimentation rate of the assembled cells increases, the system rotational rates may be increased (increasing in increments preferably of from about 1 to 2 RPM) in order to reduce the gravitationally induced orbital distortion from the ideal circular streamlines of the now increased diameter tissue pieces.

During expansion, the rotational speed of the three-dimensional TVEMF culture in the culture chamber may be assessed and adjusted so that the cells in the three-dimensional culture remain substantially at or about the horizontal axis. Increasing the rotational speed is warranted to prevent wall impact, which is detrimental to further three-dimensional growth of delicate structure. For instance, an increase in the rotation is preferred if the cells in the three-dimensional culture fall excessively inward and downward on the downward side of the rotation cycle and excessively outward and insufficiently upward on the upward side of the rotation cycle. Optimally, the user is advised to preferably select a rotational rate that fosters minimal wall collision frequency and intensity so as to maintain the three-dimensional geometry and cell-to-cell support and cell-to-cell geometry of the cells. The preferred speed of the present invention is of from about 2 to about 30 RPM, and more preferably from about 10 to about 30 RPM.

The three-dimensional culture may preferably be visually assessed through the preferably transparent culture chamber and manually adjusted. The assessment and adjustment of the three-dimensional culture may also be automated by a sensor (for instance, a laser), which monitors the location of the cells within the culture chamber. A sensor reading indicating too much cell movement will automatically cause a mechanism to adjust the rotational speed accordingly.

After the initial loading of the cell mixture and preferably the attachment phase if cell attachment substrates are utilized (2 to 4 hours) the TVEMF source is turned on and adjusted so that the TVEMF output generates the desired electromagnetic field in the three-dimensional culture in the culture chamber. The TVEMF may also preferably be applied to the three-dimensional culture during the initial loading and attachment phase. It is preferable that TVEMF is supplied to the three-dimensional culture for the length of the expansion time until the culture is terminated.

The size of the electrically conductive coil, and number of times it is wound around the culture chamber of the rotatable TVEMF bioreactor, are such that when a TVEMF is supplied to the electrically conductive coil a TVEMF is generated within the three-dimensional culture in the culture chamber of the rotatable TVEMF bioreactor. The TVEMF is preferably selected from one of the following: (1) a TVEMF with a force amplitude less than 100 gauss and slew rate greater than 1000 gauss per second, (2) a TVEMF with a low force amplitude bipolar square wave at a frequency less than 100 Hz., (3) a TVEMF with a low force amplitude square wave with less than 100% duty cycle, (4) a TVEMF with slew rates greater than 1000 gauss per second for duration pulses less than 1 ms., (5) a TVEMF with slew rate bipolar delta function-like pulses with a duty cycle less than 1%, (6) a TVEMF with a force amplitude less than 100 gauss peak-to-peak and slew rate bipolar delta function-like pulses and where the duty cycle is less than 1%, (7) a TVEMF applied using a solenoid coil to create uniform force strength throughout the cell mixture, (8) and a TVEMF applied utilizing a flux concentrator to provide spatial gradients of magnetic flux and magnetic flux focusing within the cell mixture. The range of frequency in oscillating electromagnetic force strength is a parameter that may be selected for achieving the desired stimulation of the cells in the three-dimensional culture. However, these parameters are not meant to be limiting to the TVEMF of the present invention, and as such may vary based on other aspects of this invention. TVEMF may be measured for instance by standard equipment such as an EN131 Cell Sensor Gauss Meter.

The rapid cell and tissue expansion and increasing total metabolic demand may necessitate intermittent addition of preferable components enriching the culture medium in the three-dimensional culture including, but not limited to, nutrients, fresh growth medium, growth factors, hormones, and cytokines. This addition is preferably increased as necessary to maintain glucose and other nutrient levels. During the rapid cell and tissue expansion, spent culture medium comprising waste may preferably be removed. The three-dimensional culture may preferably be allowed to progress beyond the point at which it is possible to select excellent cells orbits; at a point when gravity has introduced constraints which somewhat degrade performance in terms of a low shear three-dimensional culture.

In addition, samples of the cells in the three-dimensional culture may be collected as desired, and the culture chamber rotation may be temporarily stopped to allow practical handling. Furthermore, after expansion, the cells may be used for therapeutic purposes including for the regeneration of tissue, research, and treatment of disease.

The following example is a preferred illustration of the invention, but is not intended to limit the invention thereto.

### EXAMPLE

### Expansion of Adult Stem Cells and Hematopoietic Colony Forming Cells

Preparation

A 75 ml culture chamber of a rotatable TVEMF bioreactor, illustrated in the preferred embodiment of FIGS. 1 and 2, may preferably be prepared by washing with detergent and germicidal disinfectant solution (Roccal II) at the recommended concentration for disinfection and cleaning followed by copious rinsing and soaking with high quality deionized water. The rotatable TVEMF bioreactor may be sterilized by autoclaving then rinsed once with culture medium. If a disposable culture chamber of a rotatable TVEMF bioreactor is utilized then preferably the disposable culture chamber is already sterilized and merely needs to be removed from any packaging and assembled onto the motor and the TVEMF source of the rotatable TVEMF bioreactor.

Expansion of Adult Stem Cells

The rotatable TVEMF bioreactor may preferably be filled with culture medium consisting of Isocove's modified Dulbecco's medium (IMDM) (GIBCO, Grand Island N.Y.), supplemented with 5% human albumin, 100 ng/ml recombinant human G-CS F (Amgen Inc., Thousand Oakes, Calif.), and 100 ng/ml recombinant human stem cell factor (SCF) (Amgen). It addition, D-Penicillamine [D(−)-2-Amino-3-mercapto-3-methylbutonoic acid] (Sigma-Aldrich) a copper chelating agent, dissolved in DMSO, may preferably be introduced to the culture medium in the rotatable TVEMF bioreactor in an amount of 10 ppm. Adult stem cells from peripheral blood (CD34+/CD38−) may preferably be placed in the culture chamber of the rotatable TVEMF bioreactor at a concentration of 0.75×10⁶ cells/ml. Preferably, the culture chamber is equilibrated before the cell mixture is placed therein. If a culture medium flow loop is utilized, as depicted in the preferred embodiment in FIG. 3, then equilibration of the culture medium is also preferable to create a stabilized culture environment. The stabilized culture environment provides for substantially low stress sheer levels for the addition of the cell mixture.

The motor should be switched on, preferably at a rate of approximately 30 RPM. If the culture chamber and culture medium therein have been equilibrated the speed of rotation should be slowly returned to the preferred rate of rotation. The TVEMF source may also preferably be turned on to the preferred gauss and oscillating range.

The expansion may preferably progress for seven days without any exchange of culture medium or removal of waste. Control samples should be expanded in a rotatable bioreactor without exposure to a TVEMF during the expansion process. The controls should be expanded under the same conditions as the samples in the rotatable TVEMF bioreactor and for the same amount of time.

After the expansion is terminated, the cell viability should preferably be assayed and the number of cells determined and compared with that of the controls. It is expected that ultimately, the cells expanded in the rotatable TVEMF bioreactor number per volume at least seven times as many as those that are grown in the rotatable bioreactor without exposure to a TVEMF. The viability of the cells expanded in both the rotatable TVEMF bioreactor and the rotatable bioreactor are expected to preferably range from about 98% to about 100%.

Essentially the same procedure may preferably be followed for expanding the following hematopoictic colony forming cells: Colony-forming unit lymphocyte (CPU-L), Colony-forming unit erythrocyte (CFU-E), Colony-forming unit granulo-monocyte (CFU-GM), Colony-forming unit megakaryocyte (CFU-Me) with preferred conditions indicated below according to the cell types.

Expansion of Hematopoietic Colony-Forming Cells

Hematopoictic colony forming cells may preferably be cultured (1×10⁵ cells/ml) in 0.8% methylcellulose with IMDM, 30% fetal calf serum, 1.0 U/ml erythropoietin (Amgen), 50 ng/ml recomnbinant human GM-CSF (Immuniex Corp., Seattle Wash.), and 50 ng/ml SCF (Amgen). 1 ml aliquots of each culture mixture may preferably be placed in a 75 ml rotatable TVEMF bioreactor already filled with culture medium. The rotatable TVEMF bioreactor may preferably be placed in a 37° C. air humidifier in 5% CO₂.

Expansion may preferably be allowed to proceed for 7 days without any exchange of culture medium (or supplements), cell sampling, and viability assaying. All cultures may preferably be analyzed after 7 days of incubation for the number of burst-forming unit-erythroid (BFU-ES) colonies (defined as aggregates of more than 500 hemnoglobinized cells or 3 or more erythroid subcolonies). The cultures may also preferably be analyzed for the number of CFU-GM colonies of granulocytic or monocyte-macrophage cells or both, for the number of CFU-granylocyte-crythroid-macrophage cells or both, and for the number of CFU-granylocyte-erythroid-macrophage-megakaryocyte (CFU-GEMM) containing all elements. The analysis procedure is preferably performed by FACScan flow cytometer (Becton-Dickinson) equipped with CELLQuest software (Becton Dickinson).

Preparation and Analysis of Cells

Lymphocytes may preferably be analyzed by 2-color staining using the following antibody combinations: CD56+CD16-PE/CD3-FITC, CD3-PE/CD4-FITC, CD3-PE/CD-FITC, CD19-PE. Controls include IgG1-PE/IgG1-FITC for isotype and CD14-PE/CD45-FITC for gating. Adult stem cells may preferably be analyzed by 3-color staining with the fluorochromes PerCP/PE/FITC using the following antibody combinations: CD45/CD90/CD34, CD45/CD34/CD38, CD45/CD34/CD33, and CD45/CD34/CD15. CD45/IgG1/IgG1 used as a control. In brief, 1×10⁶ cells may preferably be incubated with 10:1 of antibodies at 2-8° C. for 15 minutes in the dark and then washed twice in phosphate-buffered saline. Then the cells should be resuspended, fixed with 1% formaldehyde, and analyzed on a FACScan flow cytometer (Becton-Dickinson) equipped with CELLQuest software (Becton Dickinson). For analyses of lymphocytes, 10,000 cells may preferably be acquired from each tube, and then gated on the basis of the forward and right angle light scatter patterns. The cutoff point should preferably be visually set at a level above background positivity exhibited by isotype controls. For analyses of adult stem cells, preferably 75,000 cells from each tube should be acquired and then sequentially gated.

Increase in Amount of Hematopoietic Colony-Forming Cells

It is expected that expansion in a rotatable TVEMF bioreactor significantly increases the numbers of hematopoietic colony-forming cells. A constant increase in the numbers of CFU-GM (above 7-fold) and CFU-GEMM (above 9-fold) colony-forming cells should preferably be observed up to day 7 with no clear plateau.

Increase in Adult Stem Cells

It is expected that expansion in a rotatable TVEMF bioreactor significantly increases the numbers of adult stem cells. The average number of adult stem cells is expected to increase by 10-fold by day 6 and plateau on that same day. The relative number of CD34+ cells co-expressing the mycloid-lineage markers CD15 and CD33 are expected to increase significantly by days 5 and 6. After the seventh day, the cells may preferably be re-injected into the patient. The cells may preferably be injected into the bloodstream or preferably injected directly into the injured tissue.

It is expected, therefore, that rapid and significant cell expansion is accomplished by expansion in the rotatable TVEMF bioractor of the present invention, as described herein.

Various changes may be made in the invention without departing from the spirit and scope thereof and therefore the invention is not limited by that which is enclosed in the drawings and specification, including the example.

I claim:

1. A rotatable time varying electromagnetic force bioreactor comprising:
a substantially cylindrical culture chamber having rotatable chamber walls, at least one aperture, and an interior portion and an exterior portion wherein the interior portion defines a space that removably receives a cell mixture;
an electrically conductive coil wrapped around the exterior portion of the culture chamber;
a motor connected to the culture chamber to rotate the culture chamber about a substantially horizontal axis; and
a time varying electromagnetic force source operatively connected to the electrically conductive coil.

2. A time varying electromagnetic force rotatable bioreactor as in claim 1, wherein the electrically conductive coil is a solenoid.

3. A time varying electromagnetic force rotatable bioreactor as in claim 1, wherein the electrically conductive coil is substantially cylindrical.

4. A time varying electromagnetic force rotatable bioreactor as in claim 1, wherein the electrically conductive coil has a substantially circular cross-section.

5. A time varying electromagnetic force rotatable bioreactor as in claim 1, wherein the electrically conductive coil has a substantially oval cross-section.

6. A time varying electromagnetic force rotatable bioreactor as in claim 1, wherein the electrically conductive coil has a substantially elliptical cross-section.

7. A time varying electromagnetic force rotatable bioreactor as in claim 1, wherein the electrically conductive coil is insulated.

8. A method for expanding mammalian cells comprising the steps of:
a. providing a culture chamber of a rotatable time varying electromagnetic force bioreactor;
b. filling the culture chamber with a culture medium;
c. placing a cell mixture containing mammalian cells into the culture chamber and initiating a three-dimensional culture wherein the mammalian cells have a three-dimensional geometry and cell-to-cell support and geometry essentially the same as the cells in vivo;
d. rotating the culture chamber about a substantially horizontal axis at a rotation speed while at the same time exposing the three-dimensional culture to a time varying electromagnetic force;
e. controlling the rotation of the culture chamber to maintain the three-dimensional culture; and
f. continuing rotating the culture chamber until the number of mammalian cell per volume is at least seven times greater than the number of mammalian cells in the cell mixture placed in the culture chamber.

9. The method of claim 8, wherein the three-dimensional culture has the properties of collocation of the culture medium and the cells, essentially no relative motion of the culture medium with respect to the culture chamber, and freedom for a three-dimensional spatial orientation.

10. The method of claim 8, wherein the culture medium is enriched by a culture medium flow loop comprising a supply manifold, a pump, an oxygenator, a rotatable perfusable culture chamber, and a waste.

11. The method of claim 8, wherein prior to step c., the culture medium flow loop is turned on.

12. The method of claim 8, wherein the culture medium flow loop enriches the culture medium with at least one selected from the group consisting of growth factors, cytokinies, hormones, oxygen, nutrients, acids, bases, buffers, and fresh culture medium prior to entering the rotatable perfusable culture chamber.

13. The method of claim 8, wherein the mammalian cells are human.

14. The method of claim 8, wherein the mammalian cells are selected from the group consisting of adult stem, mesenchymal, heart, liver, hematopoictic, skin, muscle, intestinal, pancreatic, central nervous system, cartilage, connective pulmonary, spleen, bone, and kidney.

15. The method of claim 8, wherein the time varying electromagnetic force has a force amplitude less than 100 gauss and slew rate greater than 1000 gauss per second.

16. The method of claim 8, wherein the time varying electromagnetic force has a substantially low force amplitude bipolar square wave at a frequency less than 100 Hz.

17. The method of claim 8, wherein the time varying electromagnetic force has a substantially low force amplitude square wave with less than 100% duty cycle.

18. The method of claim 8, wherein the time varying electromagnetic force has slew rates greater than 1000 gauss per second for duration pulses less than 1 ms.

19. The method of claim 8, wherein the time varying electromagnetic force has a slew rate bipolar delta function-like pulses with a duty cycle less than 1%.

20. The method of claim 8, wherein the time varying electromagnetic force has a force amplitude less than 100 gauss peak-to-peak and slew rate bipolar delta function-like pulses and where the duty cycle is less than 1%.

21. The method of claim 8, wherein the time varying electromagnetic force is substantially uniformly delivered throughout the three-dimensional culture.

22. The method of claim 8, wherein the time varying electromagnetic force is applied utilizing a flux concentrator to provide spatial gradients of magnetic flux and magnetic flux focusing within the cell mixture.

23. The method of claim 8, wherein the rotation speed is of from about 2 rpm to about 30 rpm.

24. The method of claim S, wherein the rotation speed is of from about 10 rpm to about 30 rpm.

25. The method of claim 8, further comprising a step of removing toxic material from the cell mixture prior to expansion.

26. The method of claim 8, further comprising the step of removing toxic material from the three-dimensional culture after step f.

27. A composition comprising mammalian cells and an acceptable carrier prepared by the method according to claim 26.

28. A composition of claim 27, wherein the acceptable carrier is at least one of the group consisting of plasma, blood, albumin, cell culture medium, buffer and cryopreservative; and wherein the composition optionally further comprises at least one of a growth factor, a copper chelating agent, and a hormone.

29. A composition comprising mammalian cells prepared by the method according to claim 26.

30. A method of repairing tissue of a mammal comprising the step of administering to the mammal a therapeutically effective amount of a composition comprising the expanded mammalian cells of claim 26 and a pharmaceutically acceptable carrier.

31. The method of claim 30, wherein the amount of expanded mammalian cells to be administered to the mammal is at least 20 ml of a composition having 10⁷ to 10⁹ mammalian cells/ml.

32. A method of treating a disease of a mammal comprising the step of administering to the mammal a therapeutically effective amount of a composition comprising the mammalian cells of claim 26 and a pharmaceutically acceptable carrier.

33. A method of researching a disease state comprising introducing an expanded mammalian cell produced by the method of claim 8 into a test system for the disease state.

34. A method of researching a disease state comprising introducing an expanded mammalian cell produced by the method of claim 26 into a test system for the disease state.

35. The method of claim 8, wherein the culture medium further comprises at least one cell attachment substrate.
